(19) United States
(12) Patent Application Publication
(10) Pub. No.: US 2003/0166968 A1
(43) Pub. Date: Sep. 4, 2003

(54) Method of controlling the particle size in the crystallization of dimethylol alkanoic acids

(76) Inventors: Gerhard Schulz, Ludwigshafen (DE); Hagen Weigl, Ladenburg (DE); Richard van Gelder, Ludwigshafen (DE); Matthias Rauls, Limburgerhof (DE); Klaus Ebel, Lampertheim (DE)

Correspondence Address: KEIL & WEINKAUF, 1350 CONNECTICUT AVENUE, N.W., WASHINGTON, DC, 20036, US

(21) Appl. No.: 10/296,045

(22) PCT Filed: May 21, 2001
(86) PCT No.: PCT/EP01/05819

(30) Foreign Application Priority Data
May 26, 2000 (DE) 10026139.6

(51) Int. Cl. C07C 51/42

(52) U.S. Cl. 562/580

(57) ABSTRACT

Polymethylolalkanoic or monomethylolalkanoic acids of the formula (I)

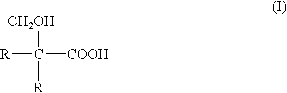

where R are identical or different and are each a methylol group or a substituted or unsubstituted aliphatic hydrocarbon radical, are selectively crystallized to give crystals having an essentially monomodal particle size distribution. This is achieved by crystallization in a temperature range from 85° C. to 50° C. and at a cooling rate of <10 K/h, giving essentially trigonally symmetric crystals.

Furthermore, polymethylolalkanoic or monomethylolalkanoic acids of the formula (I)

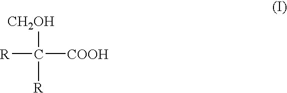

where R are identical or different and are each a methylol group or a substituted or unsubstituted aliphatic hydrocarbon radical, are selectively crystallized to give crystals having an essentially monomodal particle size distribution by carrying out the crystallization at ≦50° C. or in a temperature range from 50° C. to 5° C. and at a cooling rate of <15 K/h.

The present invention relates to a process for the selective crystallization of polymethylolalkanoic or monomethylolalkanoic acids, for example dimethylolalkanoic acids and particularly dimethylolpropionic acid.

Polymethylolalkanoic or monomethylolalkanoic acids can be prepared in various ways. For example, an aldol reaction of the appropriate aldehydes with formaldehyde can be carried out to form a polymethylolalkanal or monomethylolalkanal, followed by oxidation of this aldehyde intermediate using hydrogen peroxide (H₂O₂) or in an oxygen-containing atmosphere, as indicated by the following reaction scheme:

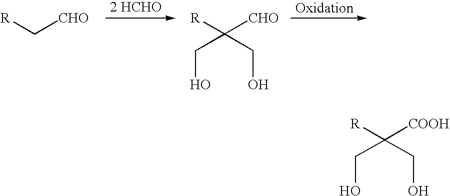

In another method, the analogous alcohol is firstly prepared in, for example, an inorganic Canizzarro reaction in which the appropriate aldehyde is reacted with excess formaldehyde in the presence of stoichiometric amounts of an inorganic base, e.g. NaOH or Ca(OH)₂. The alcohol prepared in this way, e.g. trimethylolethane or trimethylolpropane, is subsequently oxidized by means of air over heterogeneous Pd/C catalysts.

Crystallization processes for the polymethylolalkanoic or monomethylolalkanoic acids prepared in this way are also known. JP-A-11 228 489 by Nippon Kasei describes the crystallization of dimethylolalkanoic acids from water by replacement of the water by dialkyl ketones and crystallization from dialkyl ketones. Crystallization of dimethylolbutyric acid from isobutyl methyl ketone gives particles of which 83.1% by weight have a size of ≦1 mm.

In EP-A-0 937 701 by Nippon Kasei, the abovementioned process is modified in that the base still present from the aldol reaction is neutralized by means of acid and the dimethylolbutyric acid is then crystallized from an organic solvent.

In contrast to these crystallizations carried out using chemical compounds, nothing more is known about the crystallization of polymethylolalkanoic or monomethylolalkanoic acids performed by engineering means.

It is an object of the present invention to carry out the crystallization of polymethylolalkanoic or monomethylolalkanoic acids performed by engineering means, in particular that of dimethylolalkanoic acids and particularly of dimethylolpropionic acid, in such a way that essentially good filterability and a high dissolution rate of the resulting crystals is achieved.

We have found that this object is achieved when the crystallization is carried out so that a monomodal particle size distribution is obtained and the crystals have a symmetric trigonal shape. Such a monomodal particle size distribution having a high proportion of trigonally symmetric crystals is obtained according to the present invention by a process for the selective crystallization of polymethylolalkanoic or monomethylolalkanoic acids of the formula (I)

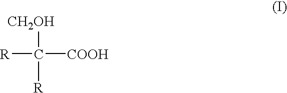

where R are identical or different and are each a methylol group or a substituted or unsubstituted aliphatic hydrocarbon radical, in which the crystallization is carried out in a temperature range from 85° C. to 50° C. and at a cooling rate of less than 10 K/h. This gives essentially trigonally symmetric crystals having a size of equal to or greater than 200 μm.

Furthermore, it has been found that an essentially monomodal particle size distribution can also be achieved by a process for the selective crystallization of polymethylolalkanoic or monomethylolalkanoic acids of the formula (I) above where R are identical, in which the crystallization is carried out at a temperature of or below 50° C. or in a temperature range from 50° C. to 5° C. and at a cooling rate of less than 15 K/h. The crystals obtained have a size equal to or less than 100 μm. They preferably have a symmetric trigonal shape.

In both the abovementioned processes according to the present invention, the monomodal particle size distribution, with or without a high proportion of trigonally symmetric crystals, is essentially achieved by avoiding a high supersaturation in the crystallization and keeping the supersaturation low. Both processes are particularly, but not exclusively, suitable for the selective crystallization of dimethylolalkanoic acids, particularly preferably dimethylolpropionic acid.

The present invention also provides for the use of the processes according to the present invention for improving the filtration properties and the dissolution properties of polymethylolalkanoic or monomethylolalkanoic acid crystals, in particular of dimethylolalkanoic acid crystals and particularly of dimethylolpropionic acid.

In the following, the invention is described in more detail with the aid of examples and with reference to the accompanying figures.

In the figures:

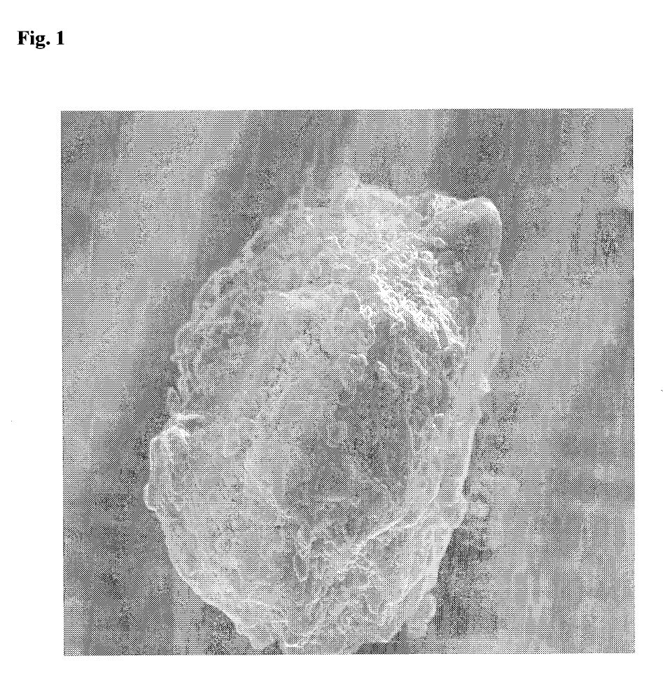

FIG. 1 shows an electron micrograph of agglomerated dimethylolpropionic acid crystals,

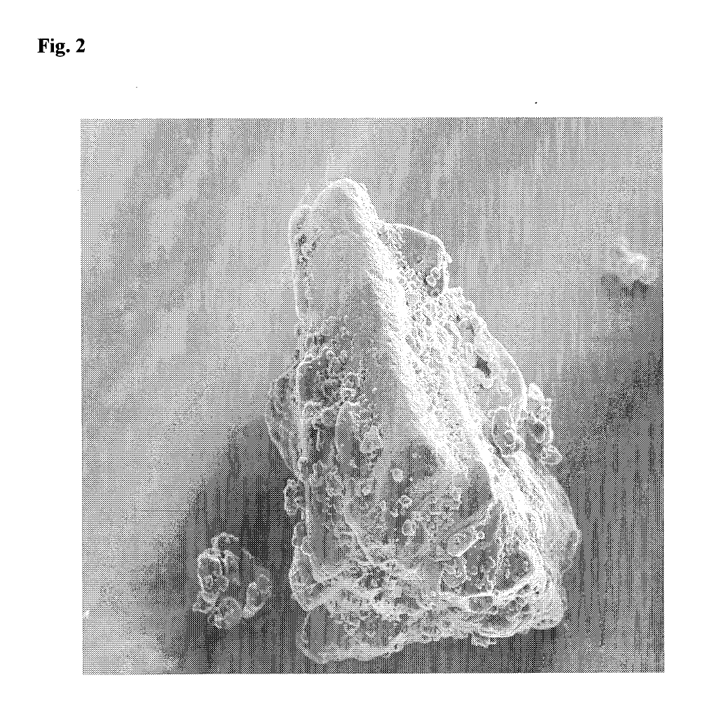

FIG. 2 shows an electron micrograph of agglomerated dimethylolpropionic acid crystals at a magnification different from FIG. 1,

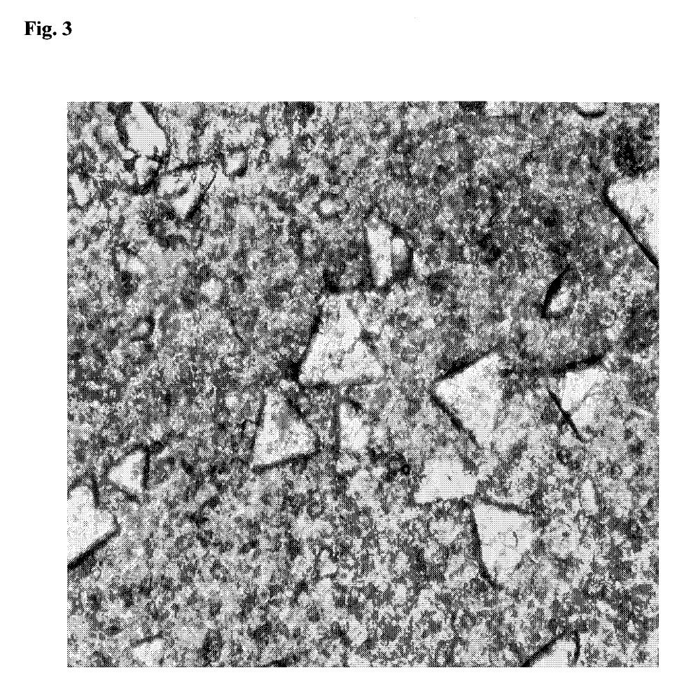

FIG. 3 shows an optical micrograph of crystallized dimethylolpropionic acid having a bimodal particle size distribution,

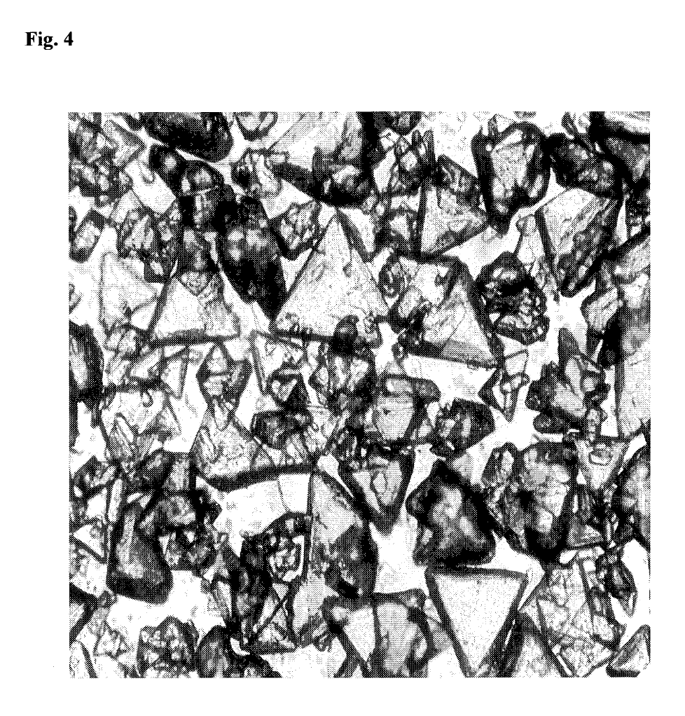

FIG. 4 shows an optical micrograph of dimethylolpropionic acid crystals having a monomodal particle size distribution,

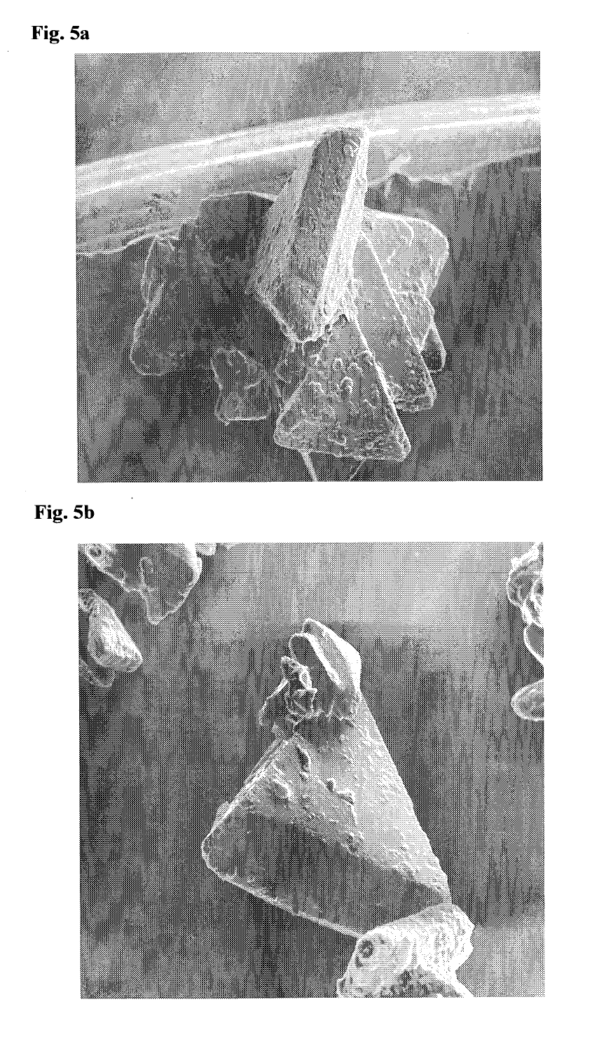

FIGS. 5a, 5b shows electron micrographs of trigonally symmetric dimethylolpropionic acid crystals having a monomodal particle size distribution,

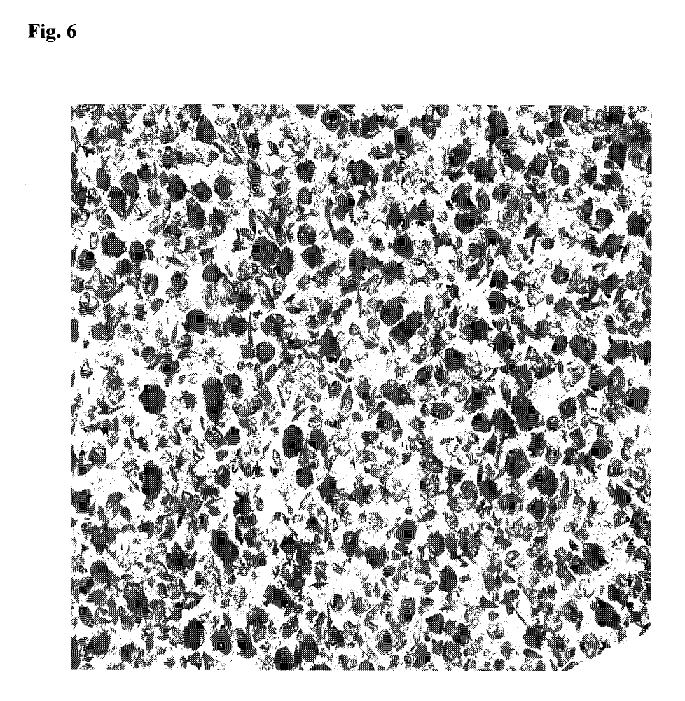

FIG. 6 shows an optical micrograph of dimethylolpropionic acid crystals without trigonally symmetric crystals,

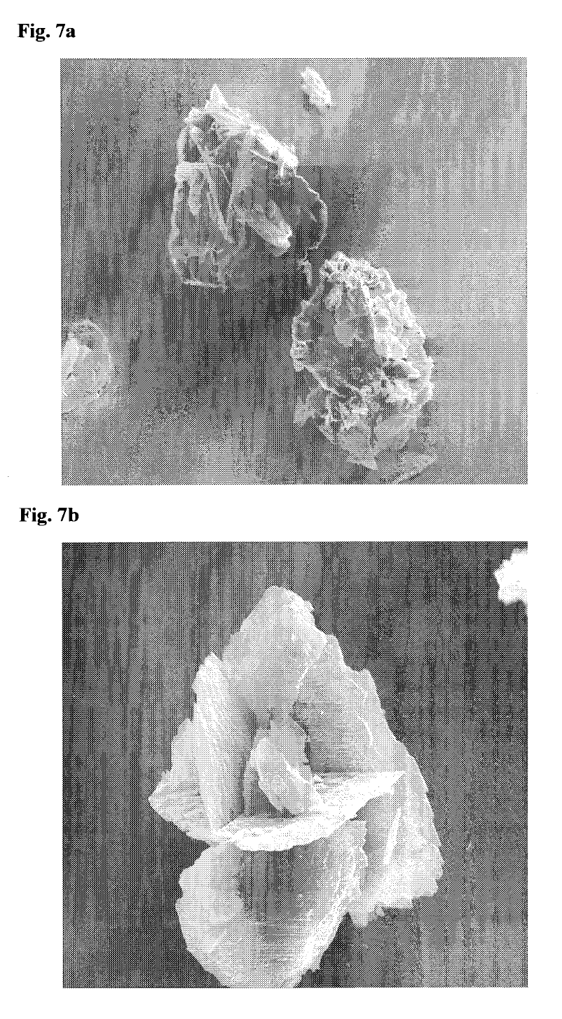

FIGS. 7a, 7b shows electron micrographs of irregularly shaped dimethylolpropionic acid crystals having a monomodal particle size distribution without trigonally symmetric crystals and

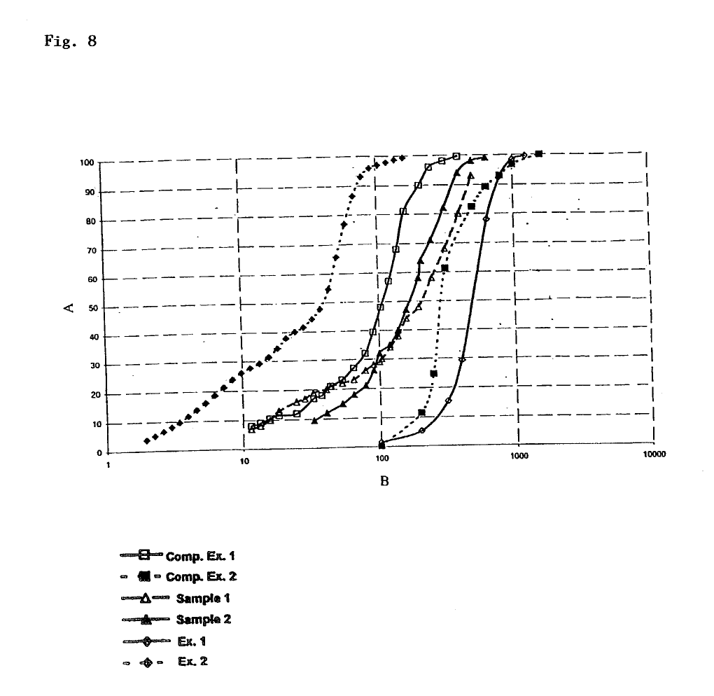

FIG. 8 shows a graph of the particle size distribution of dimethylolpropionic acid crystals obtained as described in Comparative Examples 1 and 2 and Examples 1-4. The meanings given therein are the following:

A: proportion by weight [%]

B: particle size [μm]

| | |
|---|---|
| [symbol] | Comp. Ex. 1 |
| [symbol] - | Comp. Ex. 2 |
| -Δ- | Sample 1 |
| [symbol] | Sample 2 |
| [symbol] | Ex. 1 |
| - [symbol] | Ex. 2 |

A) Preparation of Dimethylolpropionic Acid

Both the comparative examples described below and the crystallizations carried out according to the present invention are carried out using dimethylolpropionic acid by way of example. However, the crystallization processes according to the present invention are not restricted to use in the crystallization of dimethylolpropionic acid or dimethylolalkanoic acids in general, but can be applied to any polymethylolalkanoic or monomethylolalkanoic acids.

For the purposes of the examples described below, dimethylolpropionic acid was obtained by oxidation of dimethylolpropionic aldehyde using H₂O₂. The reaction mixture used for the crystallization in each case contained 37% by weight of dimethylolpropionic acid.

B) Comparative Examples

Comparative Example 1

The reaction mixture obtained in the preparation of dimethylolpropionic acid as described in A) is subjected to a batchwise crystallization by cooling and is crystallized in a temperature range from 85° C. to 5° C. at a cooling rate of 30 K/h (kelvin/hour). This gives agglomerates of large, C₃-symmetric crystals (hereinafter referred to as trigonally symmetric crystals) and irregularly shaped particles, as shown in FIGS. 1 and 2. As regards the particle size, the following product distribution was determined: 77%>50 μm, 55%>100 μm, 15%>200 μm, 1%>400 μm.

A cᵥ value of 0.71 was obtained. The filtration resistance was from 0.12 to 0.14×10¹² mPa×s/m² at a suspension concentration of 12.26% by weight.

As this example shows, crystallization occurs unselectively under the conditions chosen, giving mixtures.

Comparative Example 2

The reaction mixture obtained in the preparation of dimethylolpropionic acid as described in A) is crystallized batchwise in a temperature range from 67.5° C. to 5° C. at a cooling rate of 30 K/h. The still undried crystallized material obtained is shown in FIG. 3. In the optical micrograph shown there, the gray background consists of small crystals of irregular structure. In addition, trigonally symmetric crystals can be seen. The particle size distribution is bimodal. A filtration resistance of 3.1×10¹² mPa×s/m² was determined on this crystallized material.

C) Crystallization Process of the Present Invention

### EXAMPLE 1

The reaction mixture obtained in the preparation of dimethylolpropionic acid as described in A) is crystallized batchwise in a temperature range starting from 85° C., with the cooling rate being 5 K/h and cooling being stopped at 50° C. The resulting crystallized material comprises predominantly trigonally symmetric crystals, as shown in FIGS. 5 and 6a, 6b. About 95% of the crystals formed in this way have a particle size of >200 μm. This is shown in the graph in FIG. 7. The cᵥ value determined was 0.4.

This example shows that trigonally symmetric crystals having a particle size of predominantly greater than 200 μm can be obtained selectively in a crystallization in a selected temperature range above 50° C. Supersaturation of the reaction mixture was kept low by appropriate choice of cooling rate.

### EXAMPLE 2

The mother liquor obtained from Example 1 was cooled further at a cooling rate of 10 K/h. The crystallized material which precipitated comprised predominantly irregularly shaped crystals which were not trigonally symmetric. About 97% of the particles had a size of <100 μm. This particle size distribution, too, is shown in FIG. 7. The cᵥ value determined was 0.76.

As this example shows, crystals having a particle size of <100 μm can be obtained selectively in a crystallization below 50° C.

### EXAMPLE 3

The reaction mixture obtained in the preparation of dimethylolpropionic acid as described in A) is allowed to crystallize batchwise in a temperature range of 50° C.-5° C. at a cooling rate of 2 K/h and a stirrer power of 0.1 W/kg. Seeding is employed. The finished crystallized material contained significantly more trigonally symmetric particles than were introduced by seeding.

This example shows that trigonally symmetric crystals can also be obtained in a crystallization in a temperature range below 50° C. if supersaturation is kept low.

### EXAMPLE 4

The reaction mixture obtained in the preparation of dimethylolpropionic acid as described in A) is concentrated at room temperature by evaporation of water over 2 days to a concentration of 15% by weight. Virtually only trigonally symmetric crystals can be seen in the crystallized material.

This example, too, shows that trigonally symmetric crystals can be obtained by crystallization below 50° C. provided that supersaturation is kept low.

FIG. 8 shows the particle size distributions of dimethylolpropionic acid crystals obtained in Comparative Examples 1 and 2 and in Examples 1 and 2 in the form of a graph. The graph, too, clearly shows that the nucleation rate is increased at an increased stirrer energy, which results in the particles becoming smaller. More rapid cooling causes greater supersaturation during the crystallization, as a result of which the nucleation rate increases and smaller particles are likewise obtained.

The curves for samples 1 and 2 in FIG. 8 show the particle size of dimethylolpropionic acid products obtainable according to the prior art. Sample 1 corresponds to a commercially available product from Mallinckrodt and sample 2 originates from Perstorp AB. In both samples, the particle size distribution is in the intermediate range of FIG. 8. Neither particularly fine nor particularly large particles are obtained.

We claim:

1. A process for the selective crystallization of polymethylolalkanoic or monomethylolalkanoic acids of the formula (I) 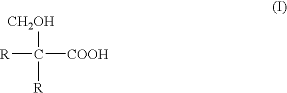
where R are identical or different and are each a methylol group or a substituted or unsubstituted aliphatic hydrocarbon radical, having an essentially monomodal particle size distribution, in which the crystallization is carried out in a temperature range from 85° C. to 50° C. and a cooling rate of less than 10 K/h and in which essentially trigonally symmetric crystals are obtained.

2. A process as claimed in claim 1, wherein crystals having a particle size of equal to or greater than 200 μm are obtained.

3. A process for the selective crystallization of polymethylolalkanoic or monomethylolalkanoic acids of the formula (I) 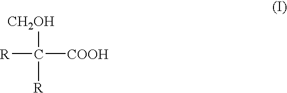
where R are identical or different and are each a methylol group or a substituted or unsubstituted aliphatic hydrocarbon radical, having an essentially monomodal particle size distribution, in which the crystallization is carried out at a temperature of or below 50° C. or in a temperature range from 50° C. to 5° C. and at a cooling rate of less than 15 K/h.

4. A process as claimed in claim 3, wherein crystals having a particle size equal to or less than 100 μm are obtained.

5. A process as claimed in claim 3 or 4, wherein essentially trigonally symmetric crystals are obtained.

6. A process as claimed in any of claims 1 to 5 for the selective crystallization of dimethylolalkanoic acids.

7. A process as claimed in any of claims 1 to 6 for the selective crystallization of dimethylolpropionic acid.

8. The use of a process as claimed in any of claims 1 to 7 for improving the filtration properties of polymethylolalkanoic or monomethylolalkanoic acid crystals.

9. The use of a process as claimed in any of claims 1 to 7 for improving the dissolution properties of polymethylolalkanoic or monomethylolalkanoic acid crystals.

10. The use as claimed in claim 8 or 9 for dimethylolalkanoic acid or dimethylolpropionic acid crystals.
